(19) United States
(12) Patent Application Publication
(10) Pub. No.: US 2019/0231846 A1
(43) Pub. Date: Aug. 1, 2019

(54) LIGHT-SENSITIVE PUMPS FOR SUPPRESSION OF CARDIAC ACTIVITY

(71) Applicants: TECHNION RESEARCH AND DEVELOPMENT FOUNDATION LTD., Haifa (IL); Rambam Med-Tech Ltd., Haifa (IL)

(72) Inventors: Udi NUSSINOVITCH, Petach Tikva (IL); Lior GEPSTEIN, Haifa (IL)

(21) Appl. No.: 16/222,091

(22) Filed: Dec. 17, 2018

Related U.S. Application Data
(62) Division of application No. 14/439,696, filed on Apr. 30, 2015, now abandoned.
(60) Provisional application No. 61/721,057, filed on Nov. 1, 2012.

(51) Int. Cl. A61K 38/16; A61K 48/00; A61N 5/06; A61K 35/33; A61K 35/34

(52) U.S. Cl. CPC A61K 38/164; A61K 38/168; A61K 48/0075; A61N 5/062; A61N 2005/0662; A61K 35/33; A61K 35/34; A61K 48/00; A61K 48/005

(57) ABSTRACT

The present invention relates to methods for attenuating cardiac activity, thereby treating cardiac disease and disorders associated with irregular or increased cardiac activity.

### CROSS-REFERENCE TO RELATED APPLICATIONS

This application is a divisional of U.S. patent application Ser. No. 14/439,696 filed Apr. 30, 2015, which is a National Phase Application of PCT International Application No. PCT/IL2013/050891, which is entitled “LIGHT-SENSITIVE PUMPS FOR SUPPRESSION OF CARDIAC ACTIVITY”, filed Oct. 7, 2013 and which claims the benefit of U.S. Provisional Patent Application No. 61/721,057, filed Nov. 1, 2012, each of which is incorporated herein by reference in its entirety.

### FIELD OF THE INVENTION

The present invention relates to methods for attenuating cardiac activity, thereby treating cardiac disease and disorders associated with abnormal increase in cardiac activity.

### BACKGROUND OF THE INVENTION

Light sensitive proteins have been demonstrated to alter neural cell excitation (Yizhar et al. Neuron, 71:9-34, 2011). Halorhodopsin (Halo), a light sensitive chloride pump, was found to cause hyperpolarization of neural cells. Archaerhodopsin-3 (Arch), a light sensitive proton pump, was found to cause cellular hyperpolarization with higher efficacy compared with different Halo protein generations. Although light-induced hyperpolarization was previously demonstrated in zebrafish, there are no reports on light induced suppression of mammalian cardiac activity.

At present, there are very limited treatments of abnormal increase in cardiac activity. Electrical defibrillation was found to have the highest efficiency in cases of ventricular arrhythmias. However, this procedure is commonly associated with complications, such as, burns, straight muscles contractions, pain, and decreased quality of life (especially in the case were shocks are being delivered inappropriately). Additionally, electrical defibrillation does not prevent the reappearance of cardiac arrhythmias. In a similar manner ablation of cardiac arrhythmias while potentially being curative in some instances is not very effective in the most common causes of cardiac arrhythmias. Moreover, ablation is associated with tissue destruction. Drug treatment for cardiac arrhythmias is also limited by the relatively low efficacy and significant proarrhythmias and other side effects associated with drug therapy, due to its global cardiac and systemic action.

There is an unmet need for therapeutic approaches of minimal invasiveness and reduced side effects which provide suppression of mammalian cardiomyocyte activation and thereby enable stable and normal cardiac activity.

### SUMMARY OF THE INVENTION

The present invention provides methods for treating heart conditions manifested in aberrant, irregular, and particularly fast and uncoordinated cardiac activities (tachyarrhythmias). Advantageously, the methods of the invention are suitable for treating the heart by transforming at one or more specific sites or areas rather than the entire heart. Furthermore, as demonstrated in the present invention, the methods of the invention are capable of correcting improper contraction of the heart by inducing a decreased, synchronous heart rate, at desired, pre-determined loci within the heart.

The present invention stems in part from the finding that the heart rate can be suppressed and maintained at proper (normal), pre-determined rates when only one or a few selected site(s) within the heart are transformed, by gene or cell therapies.

The present invention provides means for modulating cardiomyocytes excitable properties by using cell- and/or gene-therapy based on light-sensitive ion or proton pumps, and use thereof for suppressing cardiac activation. The methods of the invention may be used in the clinic for numerous applications, including by not limited to, treating different types of arrhythmias; for modulation of cardiac contractility; for blocking or filtering conduction at sites responsible for the development of arrhythmias; as a novel platform for painless, noninvasive, defibrillation for the treatment of atrial fibrillation, ventricular fibrillation, and other tachyarrhythmias. Advantageously, the methods of the invention further provide non-destructive (functional) ablation.

The present invention provides, in an aspect, a method for treating a disease or disorder associated with increased cardiac activity in a patient in need thereof, comprising the steps of introducing into at least one site of a contractile tissue in the heart of said patient a pharmaceutical composition comprising a gene encoding a light-sensitive pump; and exposing said at least one site to light, thereby suppressing said heart electrical activity in said patient.

The term “increased cardiac activity” as used herein refers to abnormally fast heart activity or irregular heart activity that includes episodes of abnormally fast heart activity.

The present invention further provides, in an aspect, a method for treating a disease or disorder associated with increased or irregular cardiac activity in a patient in need thereof, comprising the steps of introducing into at least one site of a contractile tissue in the heart of said patient a pharmaceutical composition comprising at least one cell transfected with a gene encoding a light-sensitive pump; and exposing said at least one site to light, thereby suppressing the heart electrical activity in said patient.

The present invention further provides, in an aspect, a pharmaceutical composition comprising a gene encoding a light-sensitive pump for the treatment of a disease or disorder associated with increased cardiac activity, upon exposure of said pharmaceutical composition to light after said composition is introduced into at least one site of a contractile tissue in a heart.

The present invention further provides, in an aspect, a pharmaceutical composition comprising at least one cell transfected with a gene encoding a light-sensitive pump for the treatment of a disease or disorder associated with increased or irregular cardiac activity upon exposing said pharmaceutical composition to light after being introduced into at least one site of a contractile tissue in a heart of said patient.

In some embodiments, said suppressing of said heart electrical activity comprises inducing cardiomyocytes hyperpolarization. It should be noted that suppressing the electrical activity of the heart refers to inducing normal and stable heart activity.

In some embodiments, said disease or disorder is selected from the group consisting of tachyarrhythmia, cardiac arrhythmia, malignant arrhythmia and ventricular arrhythmia. Each possibility represents a separate embodiment of the present invention.

In some embodiments, said light-sensitive pump is a light-sensitive proton pump or a light-sensitive chloride pump. Each possibility represents a separate embodiment of the present invention.

In some embodiments, said light-sensitive pump is selected from the group consisting of archaerhodopsin-3, halorhoropsin, e-bacteriorhodopsin (eBR) and eNpHR3.0. Each possibility represents a separate embodiment of the present invention.

In some embodiments, the light sensitive pump is archaerhodopsin-3 and the light is in a wavelength within the range of 550 nm to 580 nm.

In some embodiments, the light sensitive pump is eBR and the light is in a wavelength within the range of 520 nm to 560 nm.

In some embodiments, the light sensitive pump is eNpHR3.0 and the light is in a wavelength within the range of 570 nm to 610 nm.

In other embodiments, said light-sensitive proton pump is archaerhodopsin-3 or an active variant, derivative or fragment thereof. Each possibility represents a separate embodiment of the present invention.

In yet other embodiments, said light-sensitive chloride pump is halorhoropsin or an active variant, derivative or fragment thereof. Each possibility represents a separate embodiment of the present invention.

In some embodiments, exposing the at least one site to light, hyperpolarizes a plurality of cells in said at least one site.

In some embodiments, said at least one site at said contractile tissue is selected from the group consisting of the myocardial apex, the apical region of the heart, the sinoatrial node, the atrioventricular node, the left bundle branch, the right bundle branch, the right atrium, the left atrium, the right ventricle and the left ventricle. Each possibility represents a separate embodiment of the present invention.

In some embodiments, said exposing comprises exposing a plurality of sites to light.

In certain such embodiments, said plurality of sites is exposed to light simultaneously.

In certain such embodiments, said plurality of sites is exposed to light consecutively.

In other certain such embodiments, some parts of said plurality of sites are exposed to light simultaneously, while other parts of said plurality of sites are exposed to light consecutively. Each possibility represents a separate embodiment of the present invention.

In some embodiments, said light has a wavelength within the range of 500-700 nm. In some embodiments, said light has a wavelength is within the range of 520 nm to 610 nm.

In some embodiments, said light is delivered at an intensity of at least 7 mW/mm².

In some embodiments, said light is a flashing light.

In some embodiments, said flashing light is delivered at a frequency ranging from 60 to 300 flashes/min.

In some embodiments, said frequency is lower than 200 flashes/min.

In some embodiments, said duration of each flash of said flashing light is at least 1 ms.

In some embodiments, said cell is selected from the group consisting of fibroblasts, cardiomyocytes and stem cells derivatives. Each possibility represents a separate embodiment of the present invention.

In some embodiments, said at least one cell is an autologous cell derived from said patient.

In some embodiments, said at least one cell is capable of electronic coupling or fusing with said contractile tissue of the heart of said subject in need thereof. Each possibility represents a separate embodiment of the present invention.

In yet another aspect, the present invention provides a kit for treating a disease or disorder associated with abnormal increase in cardiac activity, the kit comprising a pharmaceutical composition comprising at least one cell transfected with a gene encoding a light-sensitive pump; and means for facilitating coupling or fusing said at least one cell with a contractile tissue of a subject in need thereof.

In some embodiments, the kit further comprising a light source, wherein the light source is adapted for providing at least one of light at a wavelength within the range of 500 nm to 700 nm, light at a wavelength within the range of 520 to 590 nm, flashing light, flashing light ranging from 60 to 300 flashes/min, light at an intensity of at least 7 mW/mm² and flashing light with a duration of at least 1 ms for each flash.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 Box-plots of the contraction rate of beating neonatal rat (Sprague-Dawley) ventricular cardiomyocytes (NRCM) co-cultured with HEK293 cells transfected with the Arch gene (HEK-Arch) cells prior to illumination (left), during illumination (middle) and following illumination (right). Measurements of each period was conducted throughout 30 sec. Matched pairs (corresponding to the same culture) are inter-connected by a line. The black dots represent raw values. In these and subsequent box plots, the central line represents the distribution median; the box spans from 25 to 75 percentile points.

FIGS. 2A-2J Monolayer of neonatal rat cardiomyocytes co-cultured with HEK-Arch cells (FIG. 2A). The black circle represents the area on which the light-emitting diode (LED) illumination was focused. The presence of the Arch protein is indicated by green fluorescence (FIG. 2B). Activation maps of spontaneous contraction prior to illumination (FIG. 2C), the first (FIG. 2D), the second (FIG. 2E), the third (FIG. 2F), and the fourth (FIG. 2G) contractions following illumination, following 30 seconds of illumination (FIG. 2H), the first contraction following termination of illumination (FIG. 2I), and 30 seconds following the termination of illumination (FIG. 2I).

FIGS. 3A-3D NRCM and HEK-Arch cells co-culture. Three electrodes are marked by circles denoted 1, 2 and 3 (FIG. 3A). Illumination is focused on the electrode #1. Electrical activity is measured at darkness (FIG. 3B), during LED illumination (FIG. 3C), and following termination of illumination at complete darkness (FIG. 3D).

FIGS. 4A-4D Embryonic body of cardiomyocyte driven human embryonic stem cells, co-cultured with HEK-Arch cells on 60-electrodes microelectrode array (MEA) (FIG. 4A), as indicated by green fluorescence (FIG. 4B). Raw data of electrical activity (FIG. 4C) and contraction rate display (FIG. 4D) is presented.

FIGS. 5A-5C shows NRCMs co-culture (FIG. 5A), where the presence of ChR2 is noted by the presence of GFP corresponding to dark grey background (FIG. 5B), and ArchT is detected by red (dark background) fluorescence (FIG. 5C).

FIGS. 6A-6C show complete termination of activation of NRCMs transfected with ArchT during illumination with 624 nm LED (FIG. 6A). Stable pacing was provided to NRCMs suppressed by illumination with 624 nm LED via flashing ChR2-containing NRCMs with 470 nm at 100 flash/min. (FIG. 6B) and 150 flash/min. (FIG. 6C).

### DETAILED DESCRIPTION OF THE INVENTION

The present invention provides, in an aspect, a method for treating a disease or disorder associated with increased or irregular cardiac activity in a patient in need thereof, comprising the steps of introducing into at least one site of a contractile tissue in the heart of said patient a pharmaceutical composition comprising a gene encoding a light-sensitive pump; and exposing said at least one site to light; thereby suppressing said heart electrical activity in said patient.

The present invention further provides a pharmaceutical composition comprising a gene encoding a light-sensitive pump for the treatment of a disease or disorder associated with increased cardiac activity, upon exposure of said pharmaceutical composition to light after said composition is introduced into at least one site of a contractile tissue in a heart.

The present invention further provides a pharmaceutical composition comprising at least one cell transfected with a gene encoding a light-sensitive pump for the treatment of a disease or disorder associated with increased or irregular cardiac activity upon exposing said pharmaceutical composition to light after being introduced into at least one site of a contractile tissue in a heart of said patient.

The present invention further provides the use of a pharmaceutical composition comprising a gene encoding a light-sensitive pump for the treatment of a disease or disorder associated with increased cardiac activity, upon exposure of said pharmaceutical composition to light after said composition is introduced into at least one site of a contractile tissue in a heart.

The present invention further provides the use of a pharmaceutical composition comprising at least one cell transfected with a gene encoding a light-sensitive pump for the treatment of a disease or disorder associated with increased or irregular cardiac activity upon exposing said pharmaceutical composition to light after being introduced into at least one site of a contractile tissue in a heart of said patient.

The term “pharmaceutical composition” as used herein refers to a composition comprising at least one active ingredient. A gene encoding a light-sensitive channel, and a cell transfected with a gene encoding a light-sensitive channel, are each considered an active ingredient. The phrase “introducing a pharmaceutical composition comprising a gene” as used herein refers to inserting a gene into one or more cells of the patients' contractile tissues of the heart. It should be understood that said gene can be inserted alone, or carried by a DNA vector such as a plasmid or a virus. It should be further understood that said gene can be inserted without a promoter, or cloned downstream to a constant or inducible promoter. Each possibility represents a separate embodiment of the present invention. The examples provided herein are by no way limiting to the invention, and should be used for clarification only.

The term “contractile tissue” as used herein refers to any tissue, such as, a cardiac tissue and/or a cardiac muscle tissue, containing cells that are capable of contracting or causing contraction. Each possibility represents a separate embodiment of the present invention. The contractile tissue according to the present invention, includes, but is not limited to, any section of the heart, such as, natural pacemaker cells, excluding interconnecting veins and arteries.

It would be noted that the term “exposing” refers to exposing one or more sites of the patient's heart to light, wherein each one of said one or more sites is a site that includes at least one light sensitive channel-expressing cell (e.g. cardiomyocytes or fibroblasts). In some embodiments, exposing to light refers to exposing one site at the heart. In other embodiments, exposing to light refers to exposing to light a plurality of sites at the heart. Each possibility represents a separate embodiment of the present invention. In some embodiments, said one or more sites is a site being diagnosed as a site of suppressed cardiac activity.

Illumination of light-sensitive pumps may be done internally, e.g. by an optic fiber adjacent to the heart, more specifically to said at least one site or to said plurality of sites. Illumination of these pump (i.e. within cells of the modified/transfected contractile tissue) may also be done externally, e.g. by an optic fiber attached to the patient's chest, in proximity to said at least one site or to said plurality of sites. In case of external illumination, using “red-shifted” depolarizing channels, i.e. channels activated by light of higher wavelengths, will enable greater penetration of light into the tissue and therefore the use of less light, which is important in terms of energy preservation and clinical translation.

The phrase “suppressing heart electrical activity” as used herein refers to hyperpolarizing at least a portion of the patient's heart contractile tissue, said hyperpolarization sufficient to suppress a heartbeat in said patient. It is noted that according to the teaching of the invention, suppressed heartbeat corresponds to heartbeat within the normal range of heartbeat, per age and medical statue, and not higher than the desired values. Suppressing heart electrical activity further refers to any one or more of: termination of abnormally fast cardiac activity, slowing down abnormally fast cardiac rate, and induction of a conduction block.

In some embodiments, suppressing of said heart electrical activity comprises inducing cardiomyocytes hyperpolarization.

In some embodiments, suppression of cardiac electrical activity refers to termination of the fast and uncoordinated arrhythmia (tachyarrhythmias); prevention of the fast and uncoordinated arrhythmia (tachyarrhythmias); slowing down (not terminating) the fast and uncoordinated arrhythmia (tachyarrhythmias); and filtering the electrical activity (allowing slow, not fast, rate).

The treatment can be viewed as alternative to electrical defibrillation (since it allows “painless defibrillation”), as alternative to catheter or surgical ablation for cardiac arrhythmias (since it is basically functional and non-destructive ablation, and as alternative to drugs (since drugs act globally on the heart and are therefore associated with significant side effects and low efficacy).

In some embodiments, said disease or disorder is selected from the group consisting of tachyarrhythmia, cardiac arrhythmia, malignant arrhythmia and ventricular arrhythmia. Each possibility represents a separate embodiment of the present invention. As used herein, the term “arrhythmia” is interchangeable with “cardiac arrhythmia”. In some embodiments, the terms “bradyarrhythmia” and “bradycardia” are interchangeable.

In some embodiments, said light-sensitive pump is a light-sensitive proton pump or a light-sensitive chloride pump. Each possibility represents a separate embodiment of the present invention.

In certain such embodiments, said light-sensitive proton pump is archaerhodopsin-3 or an active variant, derivative or fragment thereof. Each possibility represents a separate embodiment of the present invention. Active variant, derivative or fragment of archaerhodopsin-3, include, but are not limited to, all members of the archaeal/bacterial/fungal opsin family and any archaerhodopsin that is suitable for the method of the invention including artificial, modified and wild archaerhodopsin. Each possibility represents a separate embodiment of the present invention.

In certain such embodiments, said light-sensitive chloride pump is halorhoropsin or an active variant, derivative or fragment thereof. Each possibility represents a separate embodiment of the present invention. Active variant, derivative or fragment of halorhoropsin, include, but are not limited to, any light-gated chloride-ion channel and any halorhoropsin that is suitable for the method of the invention including artificial, modified and wild halorhoropsin. Each possibility represents a separate embodiment of the present invention.

In some embodiments, exposing the at least one site to light, hyperpolarizes a plurality of cells in said at least one site.

In some embodiments, said at least one site at said contractile tissue is selected from the group consisting of the myocardial apex, the apical region of the heart, the sinoatrial node, the atrioventricular node, the left bundle branch, the right bundle branch, the right atrium, the left atrium, the right ventricle and the left ventricle. Each possibility represents a separate embodiment of the present invention.

In some embodiments, said exposing said at least one site to light comprises exposing a plurality of sites to light.

In certain such embodiments, said plurality of sites is exposed to light simultaneously.

In certain such embodiments, said plurality of sites is exposed to light consecutively.

In other certain such embodiments, parts of said sites in said plurality of sites are exposed to light simultaneously, while others parts of said sites in said plurality of sites are exposed to light consecutively. Each possibility represents a separate embodiment of the present invention.

In some embodiments, said light has a wavelength within the range of 500-700 nm. In a certain such an embodiment, said light has a wavelength within the range of 520 to 610 nm.

Unless otherwise specified, the term “about” (or alternatively “around”) as used herein before a numerical value “X” refers to an interval extending ±30% from X, and optionally, to an interval extending ±20% from X.

In some embodiments, said light is delivered at an intensity of at least 7 mW/mm.

In order to induce multiple, consecutive heart beats, the contractile cells of the heart must be given time to contract. Thus, in some embodiments, said light is a flashing light or a pulsing light, i.e. not a constant light. In other embodiments, said light is a constant light, which is exposed to said light-sensitive channel in a flashing or pulsatile manner, e.g. by a shutter. Each possibility represents a separate embodiment of the present invention.

In some embodiments, said light is a flashing light.

In certain such embodiments, said flashing light is delivered at a frequency ranging from 60 to 300 flashes/min. In certain such embodiments, said frequency is lower than 200 flashes/min. In other certain such embodiments, said frequency is 150 flashes/min or lower. In other certain such embodiments, said frequency is 70, 80, 90, 100 or 110 flashes/min or lower. Each possibility represents a separate embodiment of the present invention.

In certain such embodiments, said duration of each flash of said flashing light is at least 1 ms. In certain such embodiments, the duration of each flash of said flashing light is 1 to 500 ms. In certain such embodiments, the duration of each flash of said flashing light is 1 to 150 ms. In certain such embodiments, the duration of each flash of said flashing light is 1 to 50 ms. Each possibility represents a separate embodiment of the present invention.

The present invention further provides, in an aspect, a method for treating a disease or disorder associated with increased or irregular cardiac activity in a patient in need thereof, comprising the steps of introducing into at least one site of a contractile tissue in the heart of said patient a pharmaceutical composition comprising at least one cell transfected with a gene encoding a light-sensitive pump; and exposing said at least one site to light; thereby suppressing said heart electrical activity in said patient.

The phrase “introducing a pharmaceutical composition comprising a cell” as used herein refers to implanting a cell in high proximity and/or in physical contact with one or more cells of the patients' contractile tissues of the heart. It should be understood that a single cell or a plurality of said cell can be implanted. Each possibility represents a separate embodiment of the present invention. The examples provided herein are by no way limiting to the invention, and should be used for clarification only.

In some embodiments, said suppressing of said heart electrical activity comprises inducing cardiomyocytes hyperpolarization.

In some embodiments, said disease or disorder is selected from the group consisting of tachyarrhythmia, cardiac arrhythmia, malignant arrhythmia and ventricular arrhythmia. Each possibility represents a separate embodiment of the present invention.

In some embodiments, said light-sensitive pump is a light-sensitive proton pump or a light-sensitive chloride pump. Each possibility represents a separate embodiment of the present invention.

In some embodiments, said light-sensitive pump is selected from the group consisting of archaerhodopsin-3, halorhoropsin, e-bacteriorhodopsin (eBR) and eNpHR3.0. Each possibility represents a separate embodiment of the present invention.

In some embodiments, said light-sensitive proton pump is archaerhodopsin-3 or an active variant, derivative or fragment thereof. Each possibility represents a separate embodiment of the present invention.

In other embodiments, said light-sensitive chloride pump is halorhoropsin or an active variant, derivative or fragment thereof. Each possibility represents a separate embodiment of the present invention.

In some embodiments, said exposing the at least one site to light, hyperpolarizes a plurality of cells in said at least one site.

In some embodiments, said cell is selected from the group consisting of fibroblasts, cardiomyocytes and stem cells derivatives.

The term “stem cells derivatives” as used herein refers to any cells derived from stems cells, including, human progenitor cells derived from pluripotent human embryonic stem cells, such as, cardiomyocytes derived from stem cells.

In some embodiments, said at least one cell is an autologous cell derived from said heart. In other embodiments, said at least one cell is an autologous cell derived from said patient in need thereof. Being autologous, cells derived from a certain patient would not raise any compatibility issues when reintroduced to the same patients' body. Thus, in some embodiments, said cell is derived from said patient.

In some embodiments, said cell is capable of electronic coupling or fusing with said contractile tissue thereby inducing hyperpolarization following light-activation of the channel.

The phrase “capable of electronic coupling or fusing” as used herein refers to the ability of the introduced light sensitive pump-transfected cells to connect, or otherwise adhere, to the patients' contractile heart cells in such a way that exposing the site of said cells to light would induce the patients' contractile heart cells to cease to contract, or to alter the contraction rate of the heart, such that, synchronization and ultimately normal heart activity, is achieved.

The terms “coupling” are interchangeable with any one or more of terms related to coupling of cells in the context of the present invention, including, but not limited to, fusing, connecting, adhering, attaching, associating with and the like.

In yet another aspect, the present invention provides a method of regulating heart activity by suppressing or inducing the activity of the heart, the method comprising:
(a) introducing into at least one site of a contractile tissue in the heart of a subject a pharmaceutical composition comprising
   (i) a gene encoding a light-sensitive pump or a cell transfected with a gene encoding a light-sensitive pump; and
   (ii) a gene encoding a light-sensitive channel or a cell transfected with a gene encoding a light-sensitive channel;
(b) and exposing said at least one site to light, wherein for inducing said heart electrical activity the light is in a wavelength within the range of 350 nm to 490 nm and for suppressing said heart electrical activity the light is in a wavelength within the range of 500-700 nm.

In some embodiments, for inducing said heart electrical activity the light is in a wavelength within the range of 450 nm to 560 nm. In some embodiments, for suppressing said heart electrical activity the light is in a wavelength within the range of 530 nm to 600 nm.

In yet another aspect, the present invention provides a kit for treating a disease or disorder associated with abnormal increase in cardiac activity, the kit comprising a pharmaceutical composition comprising at least one cell transfected with a gene encoding a light-sensitive pump; and means for facilitating transfecting the contractile tissue with said gene or means for facilitating coupling or fusing said at least one cell with said contractile tissue.

In some embodiments, the kit further comprising a light source, wherein the light source is adapted for providing at least one of light at a wavelength within the range of 500 nm to 700 nm, light at a wavelength within the range of 520 nm to 610 nm, flashing light, flashing light ranging from 60 to 300 flashes/min, light at an intensity of at least 7 mW/mm² and flashing light with a duration of at least 1 ms for each flash.

In yet another aspect, the present invention provides a kit for regulating heart activity by suppressing or inducing the activity of the heart, the kit comprising a first pharmaceutical composition comprising a gene encoding a light-sensitive pump or a cell transfected with a gene encoding a light-sensitive pump; a second pharmaceutical composition comprising a gene encoding a light-sensitive channel or a cell transfected with a gene encoding a light-sensitive channel; means for facilitating transfecting the contractile tissue with said first pharmaceutical composition or means for facilitating coupling or fusing said second pharmaceutical composition with said contractile tissue.

It is to be understood that means for facilitating coupling or fusing said at least one cell with a contractile tissue of a subject in need thereof include any means known in the art for carrying such procedure, including, but not limited to, the means exemplified hereinbelow. Furthermore, means for facilitating transfecting the contractile tissue with said gene, include any means known in the art for carrying such procedure, including, but not limited to, the means exemplified hereinbelow.

In some embodiments, the kit further comprising a first light source, wherein the first light source is adapted for providing at least one of light at a wavelength within the range of 500 nm to 700 nm, light at a wavelength within the range of 520 nm to 610 nm, flashing light, flashing light ranging from 60 to 300 flashes/min, light at an intensity of at least 7 mW/mm² and flashing light with a duration of at least 1 ms for each flash; and a second light source which is adapted for providing at least one of light at a wavelength within the range of 350 nm to 550 nm, flashing light, flashing light ranging from 60 to 300 flashes/min, light at an intensity of at least 7 mW/mm² and flashing light with a duration of at least 1 ms for each flash.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Example 1—Preparation of Cardiomyocyte Monolayers and Co-Cultures

Primary cultures of 0- to 1-day-old neonatal rat (Sprague-Dawley) ventricular cardiomyocytes (NRCM) were extracted. The tissue was suspended in a culture medium (F-10, 5% FCS, 5% horse serum, 100 U/mL penicillin, 100 mg/mL streptomycin) and cardiomyocytes were extracted enzymatically with RDB. 5-bromo-2′-deoxyuridine (BrdU) was used during the preparation of the cultures to reduce the replication of non-myocytic cells. Cells were then cultured on a microelectrode array culture plate at a density of 5-7×10⁵ cells/0.5-0.8 cm². HEK293 cells transfected with the Arch gene (HEK-Arch) were added with a ratio HEK-Arch to cardiomyocytes of 1:5-10 cells. Focused light was directed on the MEA plate while the recording of electrical activity was taking place.

### Example 2—Preparation of Cardiomyocyte Driven Human Embryonic Stem Cells (hESC) and Co-Culture

Undifferentiated hESC (A2T5 clone) were cultivated in suspension for 7 to 10 days as embryoidbodies (EBs). Beating areas, identified within the EBs after plating, were dissected and plated on 60 microelectrode array (MEA) plates (1-2 EBs per MEA plate). One to 2 days later 5×10⁴ HEK-Arch cells were added and co-cultured for 4-7 days until the beating EB was surrounded with an abundant layer of HEK-Arch cells.

### Example 3—Multielectrode Array Measurements Technique

Extracellular recordings from the cultured NRCM and from the hESC-CMs were analyzed by a microelectrode array (MEA) data acquisition system (Multi Channel Systems, Germany). The MEA consists of a matrix of 252 (16×16) or 60 (8×8) electrodes with an inter-electrode distance of 200 μm and a sampling rate of 20 kHz. Temperature was kept at 37.0±0.1° C. during measurements. Voltage was measured 4-5 days following NRCM cell culturing in order to achieve synchronized electro-mechanical activity of the contractile tissue covering the electrodes. Contraction rate was measured from the electrode which illumination was focused on. Illumination was conducted with Fiber-Coupled 1.0A monochromic LED (590 nm, Item# M590F1, Thorlab Inc.) connected to High Power LED Driver (Item# LEDD1B, Thorlab Inc.). Measurements took place during 30 sec of at complete darkness, followed by 30 sec of focused illumination with 590 nm LED, and subsequently during 30 sec of complete darkness. Measurement of electrical activity from EBs was conducted 4-7 days following co-culture with HEK-Arch cells.

Data Analysis. The 252-channel data from the MEA recording were analyzed by custom-made Matlab based software. Local activation time (LAT) was calculated by detecting the local maximum of the negative slope of the signal (in absolute value). To reliably detect the LAT, a low pass finite impulse response filter was applied to the input signal with a pass-band frequency of 300 Hz and a stop-band frequency of 800 Hz. The LAT was detected only in regions where the ‘peak-to-trough’ amplitude of the QRS complex was larger than six standard deviations of the filtered signal. In addition, the negative slope was classified as a LAT only if it was less than a median threshold of the filtered signal minus four standard deviations of the filtered signal. Activation maps were thereafter created according to the detected LAT.

Contraction rate was measured with the peak detector utility of the MC_Rack (version 4.3.5; Multi Channel Systems). Measurement of the NRCM mean beating rate was conducted for a mean of 30 sec, prior to illumination, during illumination and following illumination. Changes in rate following illumination and termination of illumination were compared to a baseline rate measurement for 30 sec prior to illumination.

Statistical Analysis. Data were analyzed using JMP Pro version 10.0 (SAS Institute, Cary, N.C.). Results presented as mean and standard error. Matched pairs were compared with the paired t-test.

### Example 4—Multi-Electrode Array—NRCM Recording

Contraction rate of the NRCM cultures prior to illumination was 56.5±24.7 contractions/min (n=19). In all plates a complete obliteration of electrical and mechanical activity was noted during illumination (FIG. 1, p<0.001). Following termination of illumination contraction rate was restored to 55.9±15.5 contractions/min (p<0.001 compared to the rate during illumination, p>0.05 compared to the rate prior to illumination).

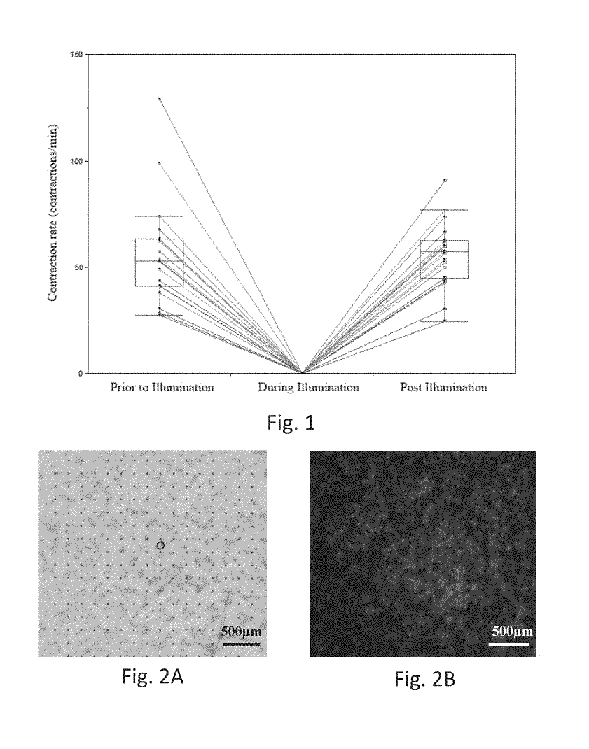

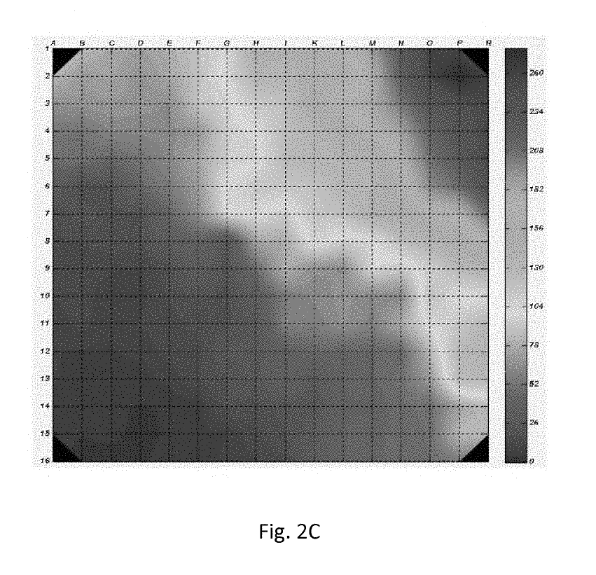

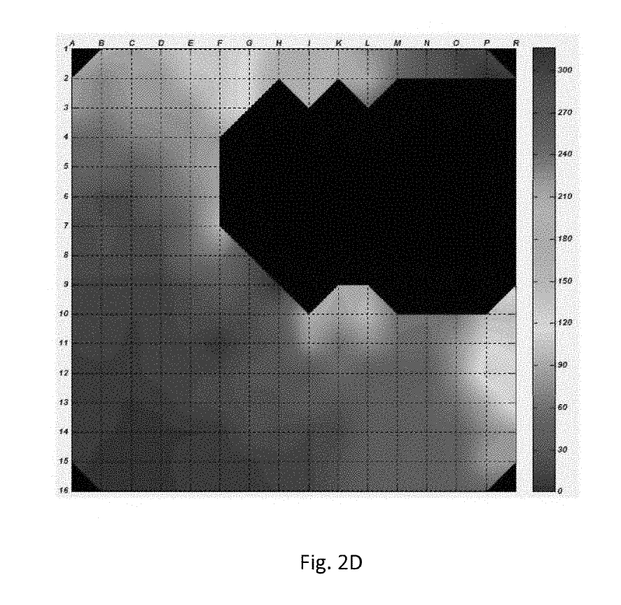

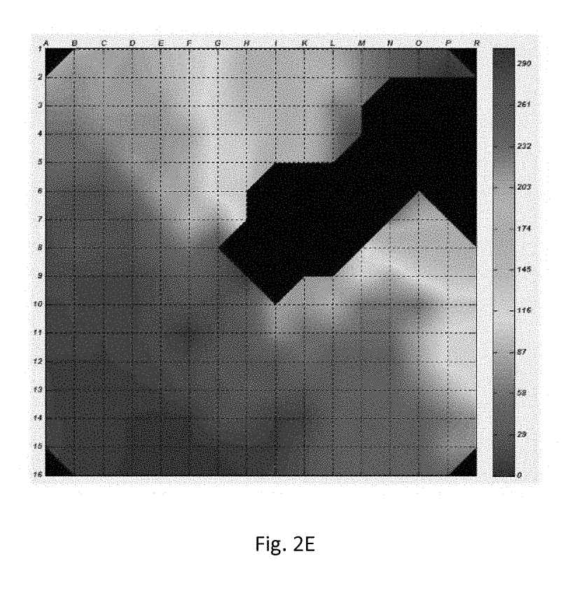

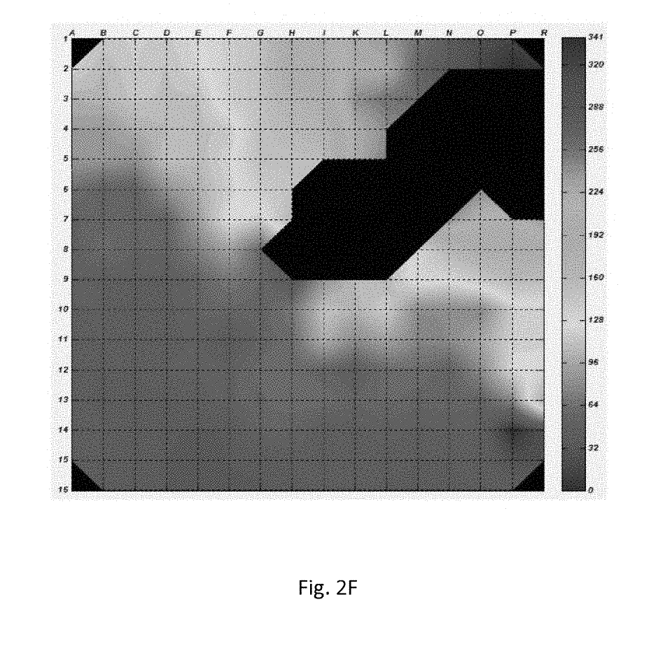

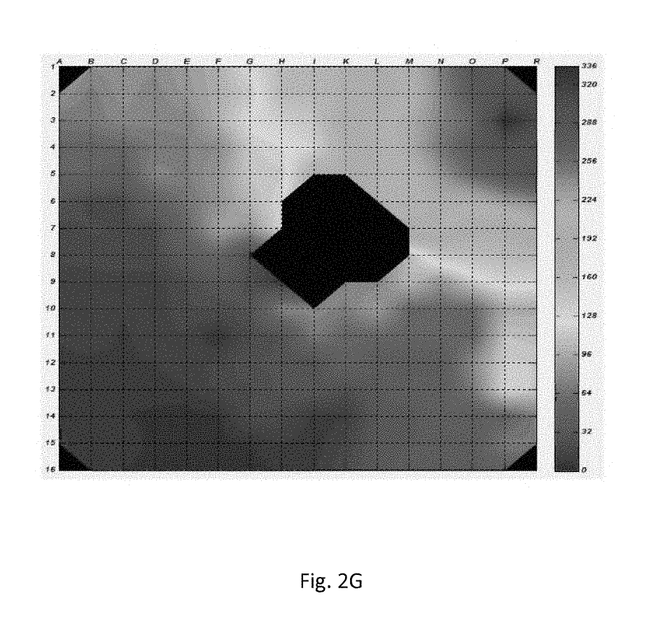

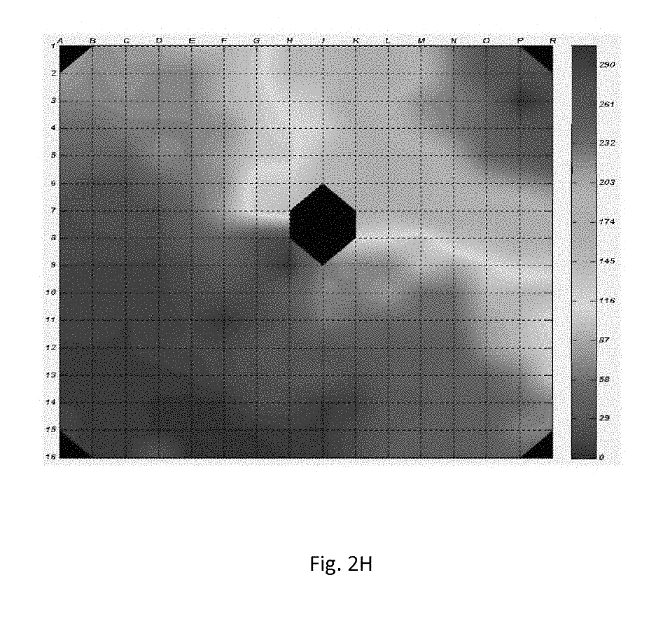

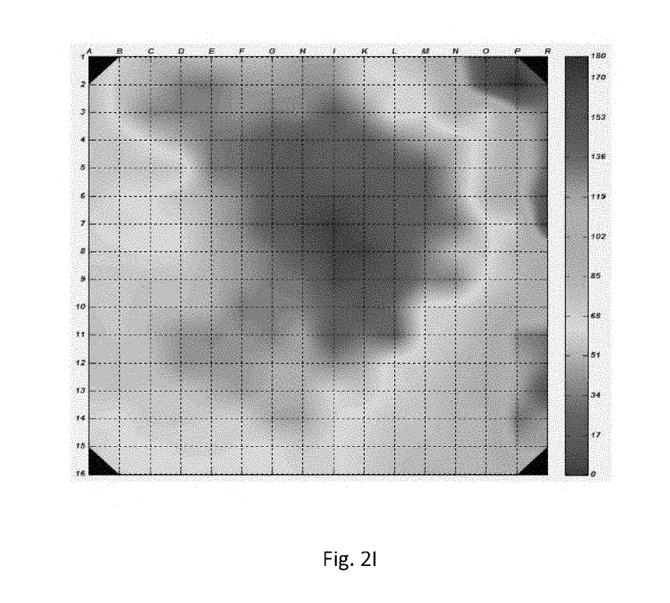

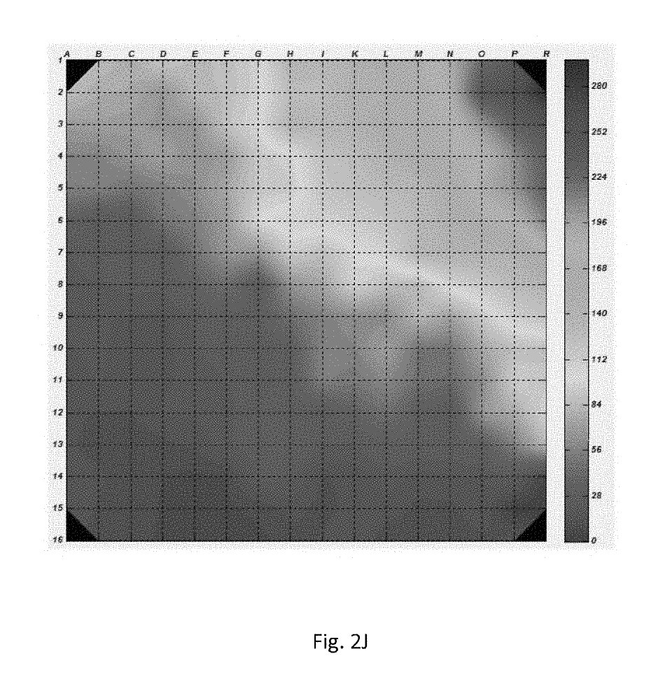

FIGS. 2A-2J demonstrates that electrical activity was completely terminated on the areas in which light was focused on for 30 sec. The affect was temporary in areas more distant from the light source (FIGS. 2C-G). Upon termination of illumination, an early contraction emerged from the silenced cells, causing activation of the monolayer (FIG. 2I). Thirty seconds following termination of illumination activation map restored to its original pattern (FIG. 2J).

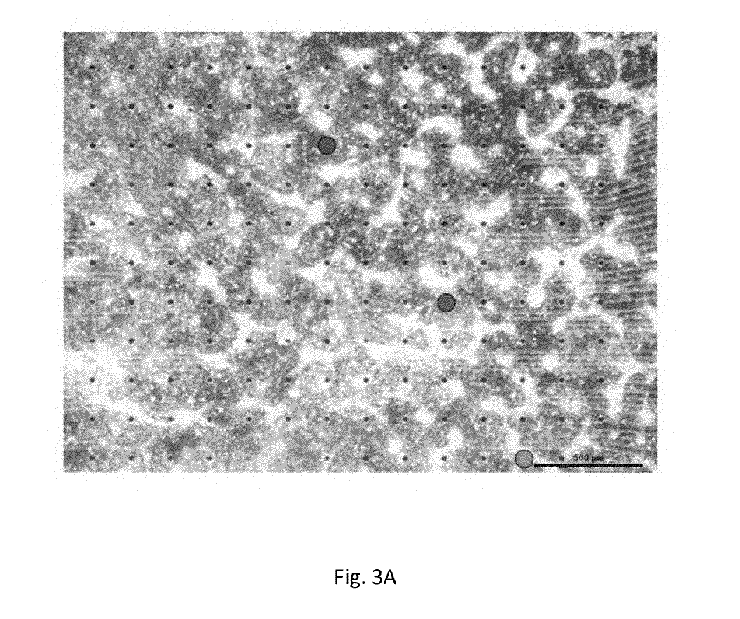

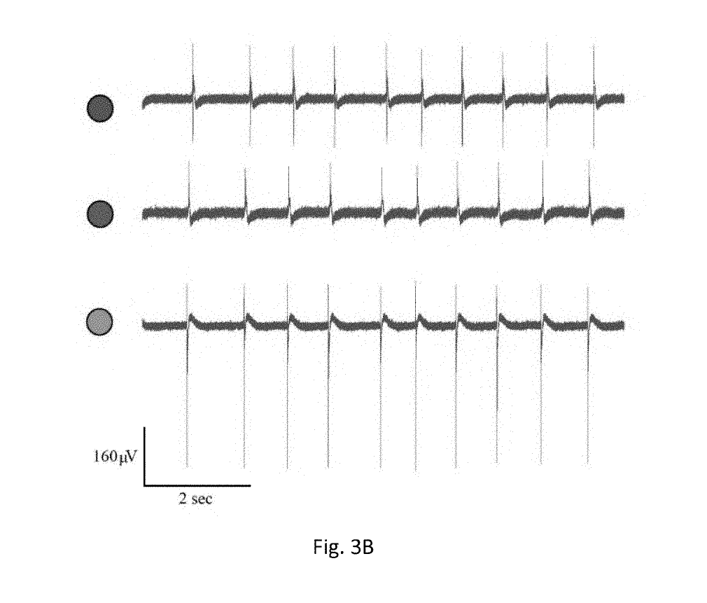

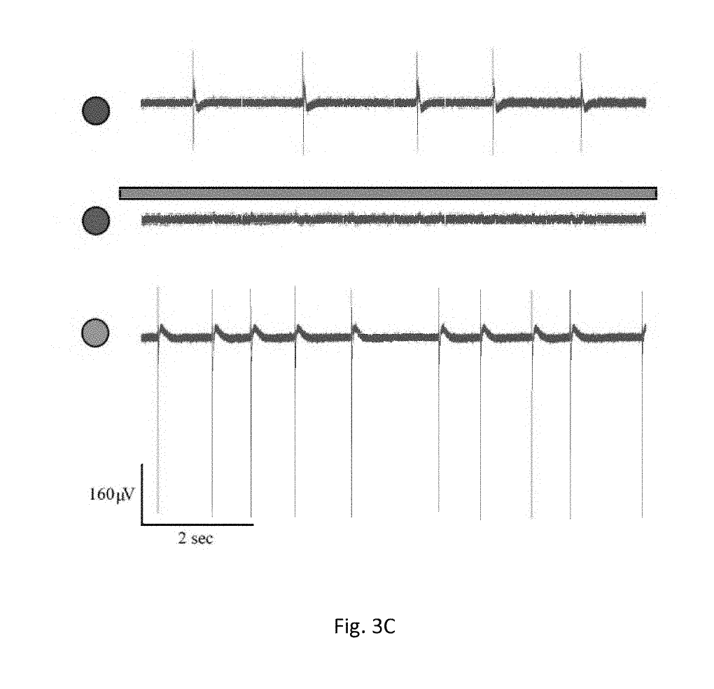

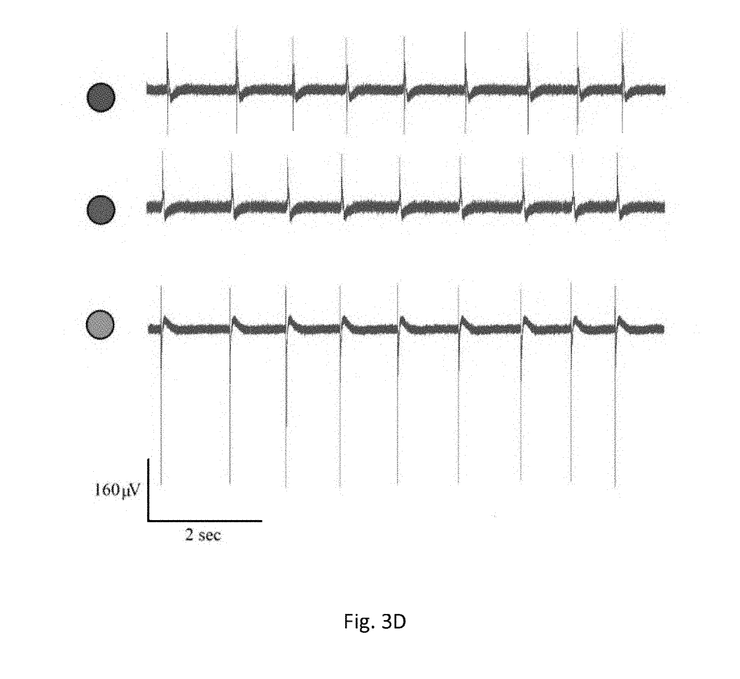

FIGS. 3A-3D demonstrates the ability of HEK-Arch cells to induce a conduction block in an electrically synchronized NRCM monolayer. Upon termination of illumination, synchronized electrical activity was restored.

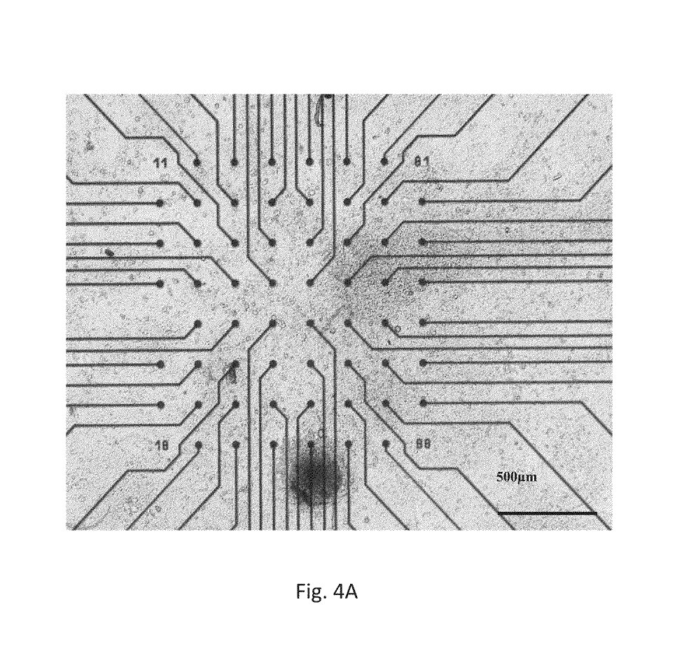

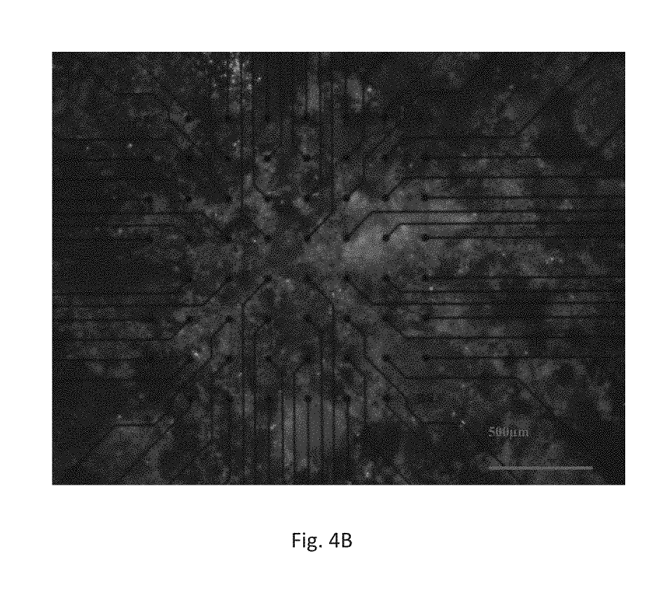

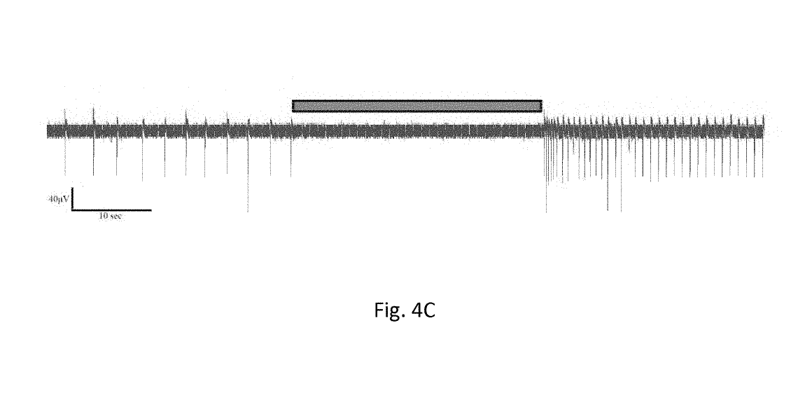

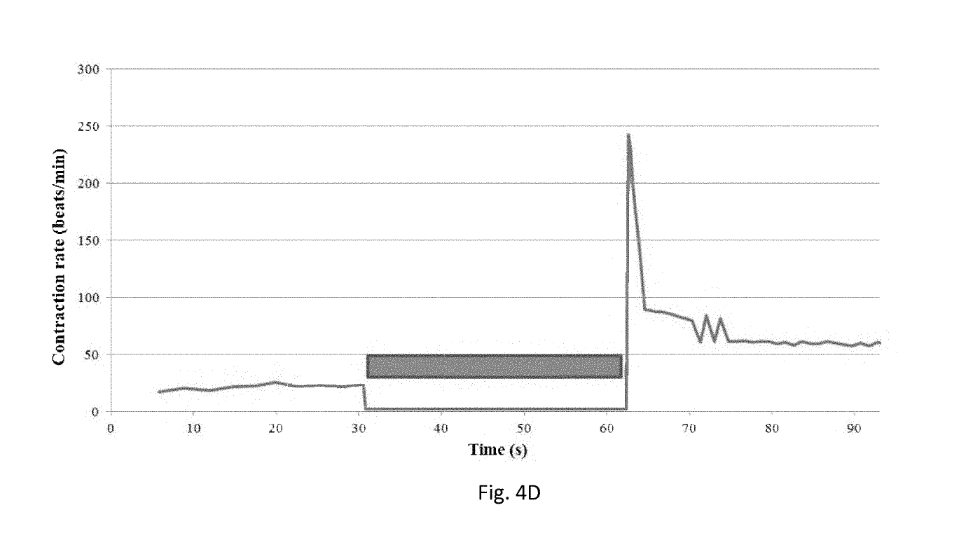

FIGS. 4A-4D demonstrate the ability of hyperpolarizing light to suppress hESCs-CMs activity during the illumination time (bar). A complete termination of electrical activity was achieved following illumination of the co-culture with 590 nm light.

### Example 5—Creation of the Engineered NIH-ChR2-ArchT Fibroblasts

The plasmids AAV-CAG-ChR2-GFP and AAV-CAG-ArchT-dtTomatato were obtained from Addgene. Stable transfection was achieved in NIH-3T3 fibroblasts with jetPEI transfection reagent. The amount of jetPEI solution mixed with the plasmid DNA resulted in an N/P ratio of 5. Thus, 3 μg of DNA and 6 μL of jetPEI were added to each well (of a six-well plate) where the fibroblasts were seeded at 50-70% confluence. Transfected cells were identified 48 h later and selected based on their fluorescence level by repeated fluorescence-activated cell sorting. Cells were grown in modified Eagle's medium (MEM) supplemented with 10%-FCS, penicillin (100 U/ml), streptomycin (100 μg/ml), 1% L-Glutamine.

### Example 6—Preparation of the NRCMs Monolayers and Co-Cultures

Primary cultures of 0 to 1-day-old neonatal rat (Sprague-Dawley) ventricular cardiomyocytes (NRCMs) were prepared as previously described. Briefly, following excision the ventricular tissue was suspended in culture-medium (Ham F-10, 5%-FCS, 5%-horse serum, 100 U/mL penicillin, 100 mg/mL streptomycin; Biological-Industries, Beit-Haemek, Israel) and enzymatically-dispersed with RDB (IIBR, Ness-Ziona, Israel). Following centrifugation, dispersed NRCMs were suspended in culture-medium and plated on microelectrode array (MEA) culture-plates, which were previously coated with fibronectin and seeded with (75-100)×10³ ChR2-ArchT-fibroblasts. The final ratio of cardiomyocytes to fibroblasts in these co-cultures was 10-16:1. The cultures were treated with 5-bromo-2′-deoxyuridine (BrdU) to reduce the proliferation of non-myocytes.

### Example 7—Microelectrode Array (MEA) Mapping

Extracellular recordings were performed using the MEA data-acquisition system (Multichannels-systems, Reutlingen, Germany). The MEA system allows simultaneous recording from 60 electrodes at a high spatial (200 μm) and temporal (15 KHz) resolution. Local activation time (LAT) at each electrode was determined by the timing of the maximal negative deflection (−dV/dtmax) of the local electrogram. This information allowed the generation of color-coded activation maps using custom-written Matlab-based software.

### Example 8—Optogenetics Illumination

Illumination of the NRCMs co-cultures was achieved with a dual band-length light source (470 nm & 624 nm, Prizmatix) and 2 mm coupled fiber-optic. The former system was equipped with an electronic shutter, which was connected to a programmable stimulus-generator (STG-1004, multichannels systems) allowing the generation of flashes (100 ms-long) at a frequency of 100-150 flashes/minute (470 nm) or 30 sec long illumination with 624 nm LED. A total of 20 consecutive flashes were given at each set of parameters.

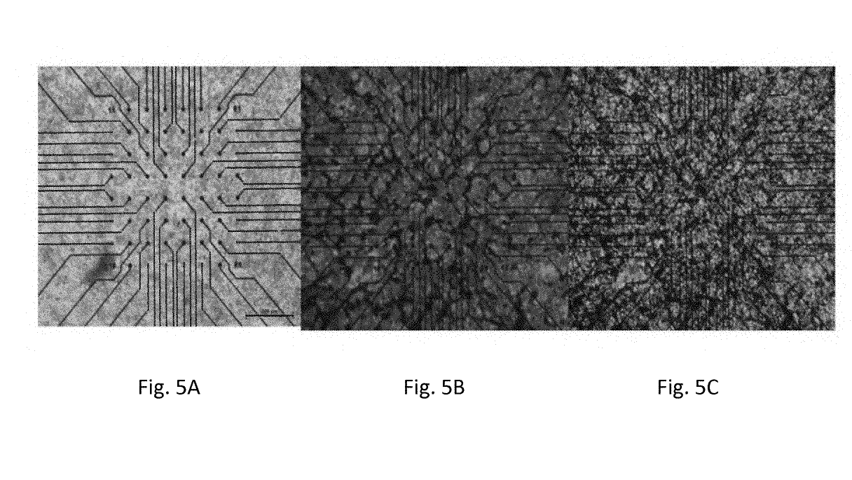

FIGS. 5A-C demonstrates the co-culture (FIG. 5A, left panel), where the presence of ChR2 is noted by the presence of GFP (FIG. 5B, middle panel), and ArchT is detected by red fluorescence (FIG. 5C, right panel).

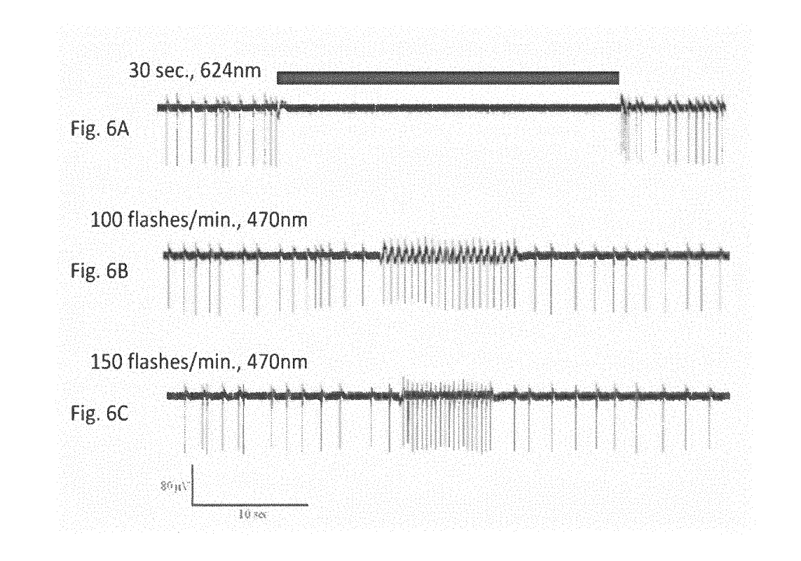

FIGS. 6A-C demonstrates complete termination of NRCMs activation during illumination with 624 nm LED (FIG. 6A) thus showing the ability to suppress heart activity using ArchT transfection and illumination at a predetermined flash rate and wavelength. FIGS. 6B and 6C demonstrate the effect of ChR2 which is also present in the transfected cell to provide a stable pacing in a condition of irregular pacing, which is a common phenomenon in patients suffering from arrhythmia. Results were repeatedly demonstrated in other cultures (N=9).

The results demonstrate the strong versatility of the methods of the invention, by implanting cells co-transfected with a light-sensitive channel and a light-sensitive pump, exposing the heart, even at a single locus, to light of a suitable wavelength can either suppress or activate the electrical activity of the heart, depending on the desired therapy.

In order to achieve induction of electrical activity, the predetermined loci which includes the transfected cells should be exposed to light within the range of 350 nm to 590 nm, or within the range of 450 nm to 560 nm. However, in order to achieve suppression of electrical activity, the predetermined loci which includes the transfected cells should be exposed to light within the range of 500 to 700 nm, for example, within the range of 520 nm to 610 nm.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method of regulating heart activity by suppressing or inducing the electrical activity of the heart, the method comprising:
a. introducing into at least one site of the heart of a subject a pharmaceutical composition comprising
i. a cell transfected with a gene encoding a light-sensitive pump; and
ii. a cell transfected with a gene encoding a light-sensitive channel; and
b. exposing said at least one site to light, wherein for inducing said heart electrical activity the light is in a wavelength within the range of 350 nm to 490 nm and for suppressing said heart electrical activity the light is in a wavelength within the range of 500 to 700 nm.

2. The method of claim 1, wherein for suppressing said heart electrical activity the light is in a wavelength within the range of 520 nm to 610 nm.

3. The method of claim 1, wherein said light-sensitive pump is selected from the The method of claim 50, wherein said light-sensitive pump is selected from the group consisting of a light-sensitive proton pump and a light-sensitive chloride pump The method of claim 1, wherein said light-sensitive channel is Channelorhodopsin-2.

5. A method of regulating heart activity by suppressing or inducing the electrical activity of the heart, the method comprising:
(a) introducing into at least one site of a contractile tissue in the heart of a subject
a pharmaceutical composition comprising
a cell transfected with:
(i) a gene encoding a light-sensitive pump; and
(ii) a gene encoding a light-sensitive channel;
(b) and exposing said at least one site to light, wherein for inducing said heart electrical activity the light is in a wavelength within the range of 350 nm to 490 nm and for suppressing said heart electrical activity the light is in a wavelength within the range of 500 to 700 nm.

6. The method of claim 3, wherein said light-sensitive proton pump is selected from the group consisting of archaerhodopsin-3 and e-bacteriorhodopsin (eBR).

7. The method of claim 3, wherein said light-sensitive chloride pump is selected from the group consisting of halorhoropsin and eNpHR3.0.

8. The method of claim 5, wherein said light-sensitive pump is selected from the group consisting of a light-sensitive proton pump and a light-sensitive chloride pump.

9. The method of claim 5, wherein said light-sensitive channel is Channelorhodopsin-2.

10. The method of claim 3, wherein said light-sensitive proton pump is selected from the group consisting of archaerhodopsin-3 and e-bacteriorhodopsin (eBR).

11. The method of claim 3, wherein said light-sensitive chloride pump is selected from the group consisting of halorhoropsin and eNpHR3.0.
